(19) United States
(12) Patent Application Publication
(10) Pub. No.: US 2010/0323386 A1
(43) Pub. Date: Dec. 23, 2010

(54) Method and apparatus to indicate combustor performance for processing chemical/biological contaminated waste

(76) Inventors: Paul M. Lemieux, Cary, NC (US); Joseph P. Wood, Cary, NC (US); Chrtstopher E. Pressley, Chapel Hill, NC (US); Richard B. Perry, JR., Zebuton, NC (US); Peter H. Kariher, Duham, NC (US)

Correspondence Address: Hendricks and Associates, P.O.Box 2509, Fairfax, VA, 22031, US

(21) Appl. No.: 12/457,847

(22) Filed: Jun. 23, 2009

(51) Int. Cl. C12Q 1/02

(52) U.S. Cl. 435/29

(57) ABSTRACT

Methods and apparatus are used for monitoring the effectiveness of a heat treatment to inactivate a contaminant in or on common building materials. The temperature is monitored or evaluated by using an internal control having a biological, chemical or electronic sensor. The sensor is bundled in common building materials to provide insulating properties so as to mimic bundles of contaminated building materials being bundled for incineration.

### GOVERNMENT SUPPORT

The work resulting in this invention was supported in part by the Environmental Protection Agency. The Government of the United States may therefore be entitled to certain rights in the invention.

### FIELD OF THE INVENTION

The present invention relates to methods and apparatus for determining the effectiveness of burning waste that has been contaminated by pathogens or toxic chemicals.

### BACKGROUND OF INVENTION

A great quantity and wide assortment of wastes are destroyed in incinerators every year in the United States. Because of the presumed heat and conditions, it has long been assumed that any pathogens would be destroyed. However, certain pathogens are resistant to heat, particularly spores, and some materials being burned may not be flammable or be incompletely burned and/or act as an insulator during the burning process, particularly when the pathogens are located inside these materials. In this situation, the wastes may be discharged from the incinerator with a residual amount of pathogens, which can potentially be harmful.

Furthermore, one is reluctant to use the final product from incinerators for any use at all or dispose of it in a landfill until some assurance is given that it does not pose harm to humans working with the material. Presently, easy methods for determining that all of the pathogens were actually inactivated or easy and accurate modeling systems for predicting that all pathogens were inactivated are unavailable.

Strips impregnated with heat resistant spores (e.g. Geobacillus stearothermophilus) have conventionally been used to determine and prove the efficacy of autoclaves used to sterilize medical equipment, supplies, fluids and microbiological media. Chemical indicators that change color upon sufficient heating have also been used to establish sterility of such materials.

In the food industry, various modeling protocols have been used to predict whether or not the sterilization procedures are sufficient to inactivate bacteria in canned foods. Stumbo, “Thermobacteriology in Food Processing”, 2^{nd} Edition, Academic Press 1973. Of particular concern in the food industry is Clostridium botulinum, which forms spores resistant to boiling temperatures and causes the deadly disease of botulism. Inside the canned environment, the material being treated is generally aqueous and not representative of incinerators.

Previously, on infrequent occasions, biological indicators have been used in incinerators to determine the efficacy of an incinerator in destroying pathogens. The State and Territorial Association on Alternative Treatment Technologies III Draft, unpublished copy of the executive summary for the conference held in Orlando, Fla. on Dec. 5-7, 2005 describes conditions to thermally destroy pathogens. Wood et al., in a paper from the International Conference on Incineration and Thermal Treatment Technologies (2005) described a study to examine thermal destruction-of biological indicators in incinerators. In this system, bacterial spores have been enclosed in a cast iron pipe and the pipe and thrown into the incinerator during processing. At the end of the burn cycle, the cast iron pipe was recovered in the bottom ash and the biological indicator removed and analyzed in a microbiological laboratory. The lack of viable bacteria indicates complete inactivation. In many cases, sufficient destruction was achieved. However, in some cases, effective destruction was not achieved in spite of the incinerators operating at acceptable temperatures.

While useful, such methodology does not accurately represent real life situations where biological contaminant spores may be enchased in insulating materials such as wallboard and ceiling tiles. By contrast, cast iron transfers heat very efficiently such that the biological indicator is more likely, to be destroyed in a cast iron pipe than in other materials.

A number of temperature sensors (e.g. thermocouples) are typically found in incinerators. However, these measure the temperature of the gases inside or perhaps the surface of an object being heat-treated. Typical temperature sensors do not measure or reflect the temperature conditions inside the materials being burned.

Devices for recording temperatures for in-situ measurements of high temperature applications are known. Datapaq sells such devices and thermal barriers for containing them, which are designed to be fed into a heated area, record and transmit temperature date wirelessly. However, these devices are quite expensive and the thermal barrier is idealized rather than reflecting actual materials being feed into the incinerator.

Experimental devices have been made for evaluating the functioning and conditions within an incinerator by traveling through it. Such devices have been used to measure bed temperatures and gas-phase species, e.g., The Ball Sampler, Martinee et al, “Development and Practical Tests of Insulating/Cooling Capsule With Sensor for In-Situ Measurements of CO Concentrations on Moving Grates in MSWI”, Proceedings of the International Conference on Incineration and Thermal Treatment Technologies, May 14-18 1007) Phoenix, Ariz. The instrumentation in these devices are typically very sophisticated and they are used to understand the conditions of the burning bed, rather than being a device used to evaluate the performance of the incinerator at destroying the materials in the bed. In addition, the sophisticated nature of the Ball samplers has made widespread routine use prohibitively expensive for routine destruction in an incinerator.

To overcome these problems, and to ensure complete inactivation of contaminants while minimizing the incineration time, the following invention was made to detect the effects of heat treatments on bundles of contaminated building material while mimicking the likely composition and arrangement of waste being treated.

### SUMMARY OF THE INVENTION

The present invention provides a way for determining the reliability of incinerator performance to process wastes contaminated with biological agents such as heat resistant Bacillus anthracis spores. This may be used in cleanup and restoration of contaminated sites.

While techniques are known for sterilizing and/or destroying varies contaminants, the present invention also seeks to determine contaminated waste destruction when the contaminant is attached to or bound within a variety of different building materials and the like. In this situation, the building materials serve as insulators delaying or preventing the heat inactivation. Also, should the building materials be bundled, e.g. tied up, bagged or piled, the insulating effect of the building materials is increased.

The present invention also provides for devices and methods for which compose an internal standard to validate the effectiveness of the heat treatment.

The present invention involves methods and apparatus, which may provide, multiple types of checks for the effectiveness of the heat treatment.

The basic steps in the present invention are bundling the heat sensitive electronics inside a highly insulated container, with the heat sensor either exposed to the incinerator environment or inserted inside building materials and subjecting the bundle to heat treatment followed by removal and testing or reading sensors or indicators of adequate heat treatment.

While the present invention is discussed in terms of destruction of heat resistant spores, the device and methods of the present invention may be applied to destruction of chemicals and contaminants on similar materials to ensure destruction thereof and prevent release of toxic agents.

### BRIEF DESCRIPTION OF THE FIGURES

FIG. 1 is a cross section of a simulated bundle containing an indicator material inside.

FIG. 2 is a cross section of a thermal protected electronic data recorder and temperature sensor.

FIG. 3 is a cross section of a combined electronic temperature recorder and a temperature sensor inside a simulated bundle.

### DETAILED DESCRIPTION OF THE INVENTION

A first preferred embodiment of the present invention is the method for using an internal standard to determine effectiveness of a heat treatment on contaminated building materials and the like. While the effectiveness of heat treatments have been determine previously, such systems were for a different environment and different type of material and for a different temperature and/or time.

The building material simulates a realistic situation in which building materials containing B. anthracis spores are removed from a building, stacked or bundled for transport, and then thrown into an incinerator still bundled along with other waste to be burned, so as to minimize handling of such materials. If the materials were bagged, the entire bag would be thrown into the incinerator with each bag containing a mix of an assortment of building materials.

Many incinerators have different temperatures at the edges and top and bottom as compared to the center of the incinerator. Furthermore, building materials are likely to be loaded in the incinerator to form a non-homogenous mass being burned. Also, additional contaminated building materials may be added when room is available when the first batch has partially or completely burned. Accordingly, it may be desirable to add multiple simulated building material bundles each to different areas or added at different times. This is particularly true for continuous processes to ensure the incineration conditions do not drift significantly.

While the present specification uses the term “building materials”, its common definition is too narrow for the purposes of the present invention. These building materials are meant to primarily be building's components after it is gutted. For example, when contamination occurs inside a building, the interior walls, flooring, ceiling and contents of the room (furniture, cloth, papers, equipment, etc. may need to be decontaminated or as in the present invention, removed and heat treated to. When the contamination occurs on the outside of the building, the exterior walls and contaminated items present outside the building would constitute the building materials.

In the specification the term “heat treatment” is intended to encompass applying sufficient heat to raise the temperature sufficiently high for a sufficient period of time to inactivate the contaminant. Typically this is performed in an incinerator and thus the terms are used interchangeably. However, depending on the composition of the materials being treated and the likely contaminant, heating under conditions that control or exclude oxygen, e.g. pyrolysis, may be beneficial. Also, depending on the contaminant and building material present, it may be beneficial to add chemicals or other components to aid in the inactivation of or immobilization of the contaminant.

Additional compositions may be added to the “heat treatment” process to produce oxidizing or reducing conditions or to produce a certain pH so that the contaminant will be more easily inactivated or so that the contaminant will not be released in gases leaving the heat treatment chamber. Also, the heat treatment may be part of cement, plaster, concrete, metal plastic or other material manufacture or recycling process. For examples, depending on the “building materials” composition concrete walls may be crushed and added to cement etc. to form into new concrete. Wallboard may be used in a process for forming Plaster of Paris and limestone may be used for cement manufacture. Plastic and metal items may be melted down and added to new or recycling manufacture processes. Cellulosic materials: wood products, composites, paper, cardboard, paneling, etc. may be pulped for a number of new products. In each of these processes, heat is applied in the manufacturing or recycling process. That heating may constitute sufficient heat treatment to inactivate the contaminant or additional heat treatment or pretreatment may be used.

In the specification the term “contaminant” is intended to encompass a number of hazardous and unwanted compositions. These include biological-containing contaminants, toxic chemical compositions, or compositions containing radionuclides.

For biological contaminants such as medical wastes, sewage sludge, corpses, slaughterhouse wastes, etc. containing microorganisms, the contaminant may be inactivated by killing the microbe, denaturing proteins, chemical alteration or burning. Bacterial spores and prions are well known to be particularly difficult to destroy by heat. Therefore, these serve as good indicators of complete inactivation of other, less hardy biological contaminants.

Chemical contaminants such as, toxic organic and inorganic compositions may also be inactivated by heat treatment under certain conditions. Organic compounds may be burned or reacted under high temperature conditions to destroy and inactivate them. Inorganic compounds may be heat treated to produce elemental forms or preferred salts, which are less toxic or more easily handled. Chemical additives to the heat treatment are preferred to aid in the destruction or conversion such as strong acids or bases. Chelating compounds and compounds that render the chemical contaminant insoluble may also be used.

For radioactive contaminants, these may be separated, immobilized or retained in a small amount of material as the remainder is burned away. Conditions should be adjusted and/or chemicals added to prevent the radioactive contaminants from being released except when so desired.

Since the present application is applicable to many contaminants, the heat treatment may be adjusted to inactivate the contaminant present, but nonetheless requires an in-situ indicator to assure that sufficient time and temperature were achieved during the heating process.

This device comprises a bundle of simulated building materials containing a temperature sensitive indicator such as a biological indicator and/or a device to measure or record temperatures within the bundle. This unit accompanies the waste through the incinerator and is recovered with the bottom ash. The unit is then opened and the temperature data and/or the biological indicator is recovered. The temperature data is then analyzed numerically and/or the biological indicator is analyzed by conventional microbiological techniques to determine spore viability. From the data, the performance of the incinerator is assessed. Data analysis techniques specific to the present invention may be used.

The temperature sensitive indicator may contain either an electrical device, chemical composition or microbiological composition or any combination of these. This indicator is then placed inside a simulated bundle of building materials (e.g. wallboard, ceiling tiles, etc.) and then fed into the incinerator or other treating system. The building materials chosen generally reflect the types of materials being treated and/or are materials that provide some insulating or buffering material. Building materials made from refractory materials which are difficult to or don't burn in an incinerator are more likely candidates for this method.

The simulated bundle of the present invention serves to provide a worst-case situation for thermal treatment conditions in the incinerator by impeding heat transfer to the temperature sensor or biological indicator. Such conservative test measures are preferred to ensure complete thermal destruction of highly lethal biological contaminants such as B. anthracis spores.

Alternatively the simulated bundle may contain a temperature-detecting device such as a thermocouple. A wire from the temperature-detecting device may extend to a temperature-recording device outside the incinerator. However, a more preferred arrangement is for the wire to extend to an electronic recorder and/or transmitter, which is contained in a highly insulated container. The electronic recorder is preferably reusable and held in such a way as to not be destroyed in the heating process. The electronic recorder in its highly insulated container may be located inside the simulated bundle or outside it provided that that the temperature-detected portion is located in the middle of the building material bundle.

Typically the electronic data recorder is highly insulated by a commercially available material such as 2-3 inches thick of Kaowool or the like. This insulation is generally greater than any insulating effects in the simulated bundle of building materials. The Kaowool may be wetted with water or other substances having a high heat of fusion or a high heat of vaporization or a high heat of decomposition to provide additional protection for the temperature data recorder. Other insulating materials such as aerogels or materials that control the temperature, such as by ablation, may be used alone or in combination. To protect the electronic data recorder from chemical damage from reactive liquids or gases in the incinerator, and to enable the bundle to be quenched with water when removed from the incinerator, the data recorder is preferably coated in a commercially available sealant and/or wrapped in an airtight bag.

After the incineration is complete in a test or actual destruction of contaminated building material, the data recorder is retrieved and the data analyzed to determine whether or not temperature conditions inside the incinerator for that particular run are sufficient to ensure complete inactivation of contaminant such as B. anthracis spores.

If the temperature sensitive indicator is composed of a biological indicator, one example is a container of heat resistant spores. After heat treatment, the container is then opened and one attempts to culture the spores. One model of the present invention uses a spore strip containing about 100,000 spores of Geobacillus stearothermophilus or Bacillus atrophaeus spores. These strips are commercially available and constitute a surrogate for Bacillus anthracis spores or Clostridium botulinum spores.

If the temperature sensitive indicator is chemical or electronic in nature, the temperature data are taken from inside the temperature detector located inside the simulate building material bundle and compared to models indicating whether sufficient thermal conditions have occurred to ensure complete inactivation of the contaminant such as B. anthracis spores. The model utilizes the thermal kinetic parameters referred to as the D- and Z-values, which are determined before the incineration but may extrapolate these to the variable and sometimes higher temperatures experienced in a building material bundle inside an incinerator.

In the present invention, the “temperature data recorder” is part of a multicomponent system, which both receives data from the temperature sensor and either records and/or retransmits the data to another electronic system for recording, analyzing, indicating and/or informing an operator of the results. The data recorder may be retrieved after heat treatment, removed from its highly insulated container and attached to a general-purpose computer or other specialized device to retransmit the data. Optionally, the data recorder may perform certain analysis and reporting operations also.

Alternatively, the data recorder may wirelessly transmit data during or after incineration. This information may be provided in real-time to provide a human or machine operator with information regarding the effectiveness of the heat treatment. This may be done by having the data recorder having a transmitter to send a signal to a receiver outside the heat treatment chamber. Depending on the design of the heat treatment chamber, an antenna may be located somewhere inside the heat treatment chamber and be connected by way of a wire to a second half of the temperature data recorder system.

Alternatively, the temperature-detecting device may be physical or chemical such as a composition that melts or decomposes at a particular temperature. Temperature detectors such as are used in automatic fire extinguishing systems and pop-up sensors may also be used. In such a system, the temperature detecting composition or device is placed inside the simulated bundle and recovered at the end of the incineration cycle, e.g. from the ash pit. An example of a suitable composition is ground glass in a metal container. Upon sufficient heating, the glass fuses together. By using a selected type of glass one can produce a temperature detector appropriate to the temperature and time conditions needed to inactivate the contaminant. Also, one can use different pigmented glass for plural types of glass and thereby determine an approximate scale of temperatures produced inside the simulated bundle by retrieving the metal container and opening it to determine which glass types have fused and which have not. Other chemical indicators change color or state, deform (change shape), break etc. once sufficient temperature has been reached for a sufficient duration.

In the variation where the simulated building material encloses a container containing a biological indicator, the biological indicator is preferably one at least as heat resistant as the contaminant. While ordinary bacteria may be used, extremophile microorganisms are particularly suitable for this purpose. Bacterial spores are a convenient indicator; well know to be resistant to harsh and high temperature conditions. Other microbes such as non-enveloped viruses and non-vegetative forms of fungi may be used.

A second preferred embodiment of the present invention is an apparatus for insertion into an incinerator in order to determine whether or not the incineration conditions are sufficient to inactivate the contaminant found or likely to be found in the building materials to be incinerated. This apparatus comprises a small container having a biological, chemical or electronic indicator of the temperature conditions. This container is embedded within a bundle of building materials. Typically, the building materials used are pieces of wallboard or ceiling tile because wallboard and ceiling tile provide thermal-insulating properties, is not made of completely flammable materials and is very inexpensive. The gypsum in the wallboard is generally a hydrate, which further absorbs heat to drive off the water in the hydrate, thereby providing even better insulating properties to the formed bundle. Ceiling tiles and other materials may be used which are inexpensive and typical of contaminated building materials.

To better simulate a bundle of building materials, fragments of comparable building materials may be bundled around the indicator/sensor and held in place by wire, a cage or the like of heat resistant material, which holds the bundle together during handling. These wires, cages etc. may also have a handle or other easy to retrieve structure for easy recovery of the biological indicator, temperature sensitive indicator or temperature sensor and/or data recorder.

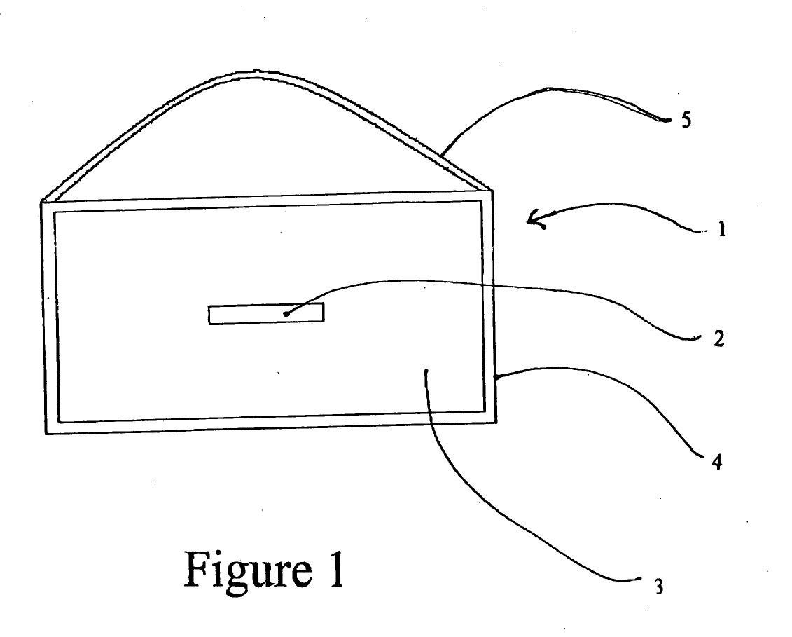

An example of the apparatus of the present invention is depicted in FIGS. 1-3. These are designed to be placed inside an incinerator and at least part not be destroyed during the heating process. In FIG. 1, the simulated bundle of building materials (1) contains a temperature sensitive indicator (2) in the middle of the simulated bundle (1). Building materials (3) or equivalents thereto are located around the temperature sensitive indicator (2) in all directions to provide insulation or protection against outside heat. The building materials (3) are held in place around the temperature sensitive indicator (2) by a wire cage (4), which is not adversely affected by heat of an incinerator. For easy removal of what remains of the simulated bundle (1) after incineration a handle (5) may be attached to the wire cage (4). As the temperature sensitive indicator (2) may be quite small and covered in a pile of ashes at the bottom of an incinerator, the wire cage (4) and handle (5) also serve for easy removal.

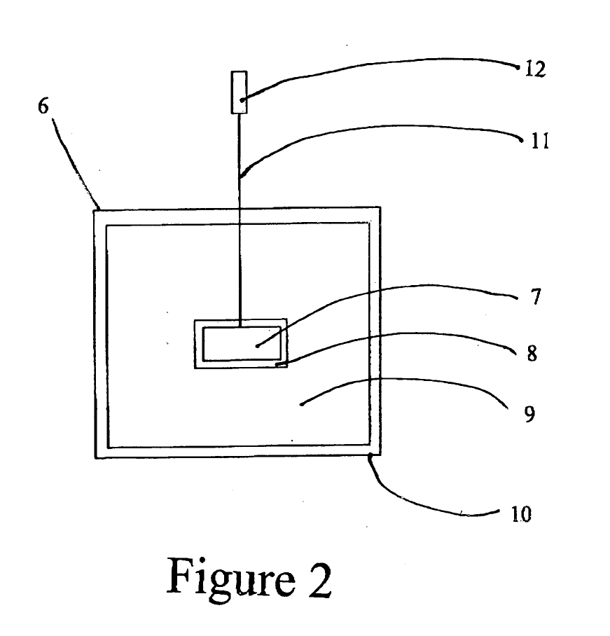

In FIG. 2, an electronic sensor and recorder system (6) is designed for use inside an incinerator. An electrical temperature data recorder (7) is sealed inside a protective sealant (8) to protect it from liquid or gaseous chemicals, which may harm the data recorder (7). A highly insulating material (9) completely surrounds the electronic data recorder (7) to protect it from the heat of the incinerator. A metal shell or box (10) surrounds the highly insulating material (9) to hold it together and protect much of the system from harmful effects of the incineration environment. A wire (11) extends from the data recorder (7) to a temperature sensor (12) located outside thermal protective walls surrounding the data recorder (7).

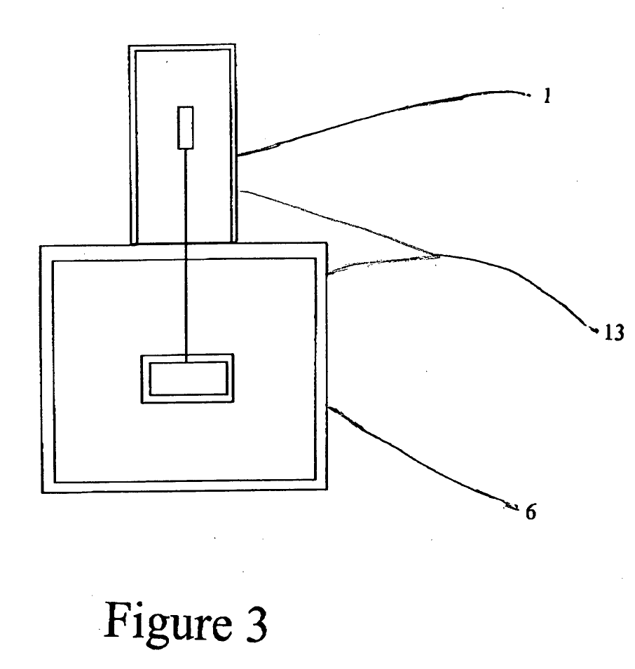

FIG. 3 shows a combined simulated bundle and electronic sensor and recorder system (13). This combined system is essentially the simulated bundle (1) of FIG. 1 located on top of the electronic sensor and recorder system (6) of FIG. 2 except for using the temperature sensor (12) instead of a temperature sensitive indicator. In this embodiment, both devices may be bound together. A temperature sensitive indicator may also be located inside the simulated bundle.

One representative example is a bundle 11 inches long, three inches wide and 3 inches in height made from pieces of wallboard 3 inches by 3 inches by ¾ inch thick. The bundle may be held together with a metal wire cage to hold the bundle together. The wire cage may have a metal handle for easy removal from the incinerator bottom ash after incineration is complete. A small container containing the indicator is located inside the bundle. As an example, the small container is a metal pipe that contains 1,000,000 spores of Geobacillus stearothermophilus.

When a temperature recorder is used, only the temperature sensor (e.g. a thermocouple) need be located inside the bundle. A wire may be connected to an electronic data recorder located elsewhere, preferably in a highly insulated container so that it is not destroyed by the temperature inside the incinerator. Alternatively, the highly insulated container containing the electronic data recorder may be located inside the simulated building material bundle but the temperature sensor will be outside the highly insulated container but inside the bundle.

Another representative size for the present invention would be an assembly of a 12×12×12 inches cube that is fed into the incinerator along with other waste material. This unit is comprised of simulated building materials along with the temperature measuring/recording device within the bundle.

A third preferred embodiment of the present invention is to feed multiple devices of the present invention into the incinerator. The devices are fed at different times, are wrapped in different simulated building materials or may be added when conditions inside the incinerator change. Furthermore, when additional materials are feed into the incinerator before the first batch is finished, that time is also suitable for adding an additional detection device of the present invention.

In a fourth embodiment, an alternative to an incinerator is used. Where one does not wish to oxidize the material, pyrolysis (no oxygen heating) is used. Also, an open fire need not be present as steam heating (under pressure or not), liquid baths of inactivating chemicals such as caustic alkali, acid, strong oxidizing or reducing agents, etc. may be used. Likewise, the contents of the heat treatment chamber may be heated by microwaves, radiant heat, convection with hot gases or general heating of the entire chamber. Multiple treatments may be used simultaneously or sequentially.

In a fifth embodiment, an alternative to heat treatment as the primary inactivator may be used. For biological contaminants, chemical and biochemical contaminants, toxins (proteinaceous or not), poisons, strong irritants etc. the inactivation technique may primarily be by chemical reaction. In this situation, a biological indicator or a chemical detecting sensor may be used in a similar manner as above for heat treatment. One example would be to use a caustic alkali (e.g. lye, lime, soda) and to have a pH sensor. Heat treatment is preferably used simultaneously. An incinerator may be appropriate or other heating with or without additional chemical treatment may be more appropriate depending on the chemical contaminant.

The present invention may also be used with a material that is not heat inactivatable or it is undesirable to heat inactivate it such as a radioactive material, a heavy metal (e.g. cadmium, lead, etc.) or toxic salt (e.g. arsenates, cyanides etc.), volatile material, etc. Depending on the specific contaminant strong oxidizing or reducing conditions may be used with or without heat to convert the material into a more stable or non-leachable form, reduce the volume of treated material and/or make the treated material appropriate for later disposal. Final disposal may be in the form of glassifying it in silicates, mixing it to form concrete or ceramic. In all of these situations, the present invention may be used to monitor and/or determine the effectiveness of the treatment conditions.

The devices of the present invention are designed inexpensively so that they may be fed into the incinerator over an arbitrary period of time to provide statistical basis for performance assurance. The bundling building materials are chosen to most closely mimic the types of building materials to be heat-treated. By having the devices be inexpensive, they may be discarded after a single use or reused, and multiple devices can be fed simultaneously to hedge against data loss due to failure of one device.

To enhance the heat treatment, one may also agitate or stir the building materials in the incinerator. This serves to break up the larger bundles and reduce the insulation effect of the building materials around the contaminated parts and if the material is being burned, to provide for a more through burn.

### Example 1

### Heat Treatment Effects on Simulated Bundles using a Biological Indicator

A biological indicator and a temperature sensor with data recorder were used on a number of different runs under differing conditions. The Biological inactivation temperature/time was correlated to the data from a thermocouple temperature sensor and mathematical modeling of the inactivation temperatures were calculated. Data and a theoretical discussion were presented in Wood et al, Environmental Science & Technology, 42(15) p. 5712-5717 (2008).

Briefly, the biological indicator was 100,000 spores of Geobacillus stearothermophilus on a strip placed in a small pipe, about 2 inches long. The pipe was placed in a bundle of 11 inches×3 inches×3 inches of drywall. Drywall pieces of 3 inches×3 inches×¾ inch were stacked with the small pipe containing biological indicator and the bundle was held in place by a mesh of 303 stainless steel. Some of these bundles were dry and some wet by being submerged in water. Similar bundles were made from ceiling tile and from carpet.

In each run, a bundle was tossed in an incinerator along with other waste at temperatures 824 degrees C. and 1093 degrees C. and retrieved at different times. The small pipe was cooled and the spore strip retrieved. Quantitative culturing of the spores was attempted and the results were considered, based on the log reduction of viable spores recovered.

All bundles had viable spores surviving for several minutes and some survived many times longer. Wet bundles provided greater resistance to heat than dry bundles and higher kiln temperatures provided a greater reduction in viable spores for otherwise identical treatment. Bundles of ceiling tile provided the most insulative effects and wallboard bundles produced viable spores even after 25 minutes. Carpet provided the least protection with the maximum time before complete inactivation of the spores being at most 9 minutes.

### Example 2

### Heat Treatment Effects on Simulated Bundles using a Thermocouple and Data Recorder

A highly insulated container of approximately an 8 inch cube was filled with wet Kaowool and a data recorder placed inside so that at least 2-3 inches coated it on all sides. The data recorder was previously coated with a sealant and placed inside a bag. A wire from the data recorder protruded from the highly insulated container and ended with a thermocouple.

Several runs were performed in the same incinerator as in Example 1. Initially the first run with the device being added to the incinerator along with other waste was with the thermocouple freely exposed to the incineration conditions. Additional runs were performed with the thermocouple placed in the same types of simulated bundles as in Example 1. Temperature measurements were recorded every 10 seconds for one hour that the device was present in the incinerator. The data was analyzed and compared to the data from the heat treatment effects data from Example 1. From this, mathematical calculations were made regarding the time and temperature conditions needed to fully inactivate the spores given different types of building materials.

It will be understood that various modifications may be made to the embodiments disclosed herein. Therefore, the above description should not be construed as limiting, but merely as exemplifications of preferred embodiments. Those skilled in the art will envision other modifications within the scope and spirit of the claims appended hereto.

All patents and references cited herein are explicitly incorporated by reference in their entirety.

What is claimed is:

1. A method for determining the effectiveness of a heat treatment to inactivate a contaminant comprising;
subjecting a simulated building material bundle, containing a temperature sensitive indicator or a temperature sensor embedded in a thermally insulating amount of building material, to an effective heat treatment to inactivate the contaminant, and
determining whether the heat treatment was effective by analyzing the temperature sensitive indicator or data from the temperature sensor.

2. The method of claim 1 wherein the contaminant is a microorganism.

3. The method of claim 1 wherein the temperature sensitive indicator is a microorganism.

4. The method of claim 1 wherein the temperature sensor provides temperature data to a data recorder.

5. The method of claim 1 further comprising subjecting multiple simulated building material bundles to the heat treatment and analyzing multiple temperature sensitive indicators or multiple temperature sensors.

6. The method for determining inactivation of a contaminant on or in building materials comprising
subjecting a) contaminated or suspected of being contaminated building materials and b) a simulated building material bundle, containing a temperature sensitive indicator or a temperature sensor embedded in a thermally insulating amount of building material, to a heat treatment,
analyzing the temperature sensitive indicator or data from the temperature sensor, and
determining whether the contaminant on or in the building materials has been inactivated by determining whether or not the temperature sensitive indicator or data from the temperature sensor indicates that the heat treatment was sufficient to inactivate the contaminant.

7. The method of claim 6 wherein the contaminant is a microorganism

8. The method of claim 6 wherein the temperature sensitive indicator is a microorganism.

9. The method of claim 6 wherein the temperature sensor provides temperature data to a data recorder.

10. The method of claim 6 further comprising subjecting multiple simulated building material bundles to the heat treatment and analyzing multiple temperature sensitive indicators or multiple temperature sensors.

11. A device for determining the effectiveness of a heat treatment to inactivate a contaminant comprising;
a simulated building material bundle, containing a temperature sensitive indicator or a temperature sensor embedded in a thermally insulating amount of building material,

12. The device of claim 11 wherein the temperature sensitive indicator is a microorganism.

13. The device of claim 11 further comprising a temperature data recorder for receiving temperature data from the temperature sensor.

14. The device of claim 13 further comprising a highly insulated container and wherein the temperature data recorder is located in a highly insulated container.

15. The device of claim 14 wherein the highly insulated container containing the temperature data recorder is located outside of the simulated building material bundle.
